(19) United States
(12) Patent Application Publication
(10) Pub. No.: US 2024/0122478 A1
(43) Pub. Date: Apr. 18, 2024

(54) PATIENT MONITORING DEVICE

(71) Applicant: KeleHealth LLC, Davie, FL (US)

(72) Inventor: Ana Kelegama, Davie, FL (US)

(21) Appl. No.: 18/231,602

(22) Filed: Aug. 8, 2023

Related U.S. Application Data
(60) Provisional application No. 63/396,107, filed on Aug. 8, 2022.

(51) Int. Cl. A61B 5/00; A61B 5/0205; A61B 5/1455; A61B 5/257; A61B 5/282; A61B 5/332; A61B 5/339

(52) U.S. Cl. CPC A61B 5/0006; A61B 5/002; A61B 5/0205; A61B 5/14552; A61B 5/257; A61B 5/282; A61B 5/332; A61B 5/339; A61B 5/1117

(57) ABSTRACT

A medical monitoring device comprises a monitoring unit. The monitoring unit comprises a processor, a transceiver in data communication with the processor, and a power supply in electrical communication with the processor and the receiver. The monitoring device further comprises a plurality of wireless electrodes. Each of the wireless electrode comprises an electrode, a transmitter in electrical communication with the electrode, a power supply in electrical communication with the transmitter, and an adhesive operably connected to the electrode. A method of generating an electrocardiogram (ECG) comprises: detecting at least 10 ECG signals through a plurality of wireless electrodes; wirelessly transmitting each of the at least 10 ECG signals from the electrodes to a monitoring unit; and displaying each of the at least 10 wireless signals on a display in real time.

### REFERENCE TO CO-PENDING APPLICATIONS

This patent application claims prior to U.S. Provisional Patent Application Ser. No. 63/396,107 filed on Aug. 8, 2022 and entitled PATIENT MONITORING DEVICE, the entire disclosure of which is hereby incorporated by reference.

### BACKGROUND

Great strides have been made in the development of therapeutics and medical devices for treating patients. However, much progress needs to be made in the development of patient monitoring equipment that can be used for monitoring and diagnosing patients in a clinical setting.

For example, electrocardiogram (ECG) monitors typically have a plurality of electrodes attached to a patient, sometimes as many as 5 or more electrodes. They all have leads or wires connecting them to a monitoring device, which in turn is typically connected to the hospital network with an Ethernet connector. This arrangement can present a multitude of problems. For example, the leads are uncomfortable and interfere with movement of the patient. They also tether a patient to their hospital bed and room. The leads have to be disconnected whenever a patient is moved to other rooms or apartments for testing, examination, or treatment. Additionally, the wires can become accidently disconnected resulting in a loss of monitoring. Monitoring agitated and confused patients is even more challenging because they may pull on and intentionally disconnects ECC leads themselves. Another problem is the number of monitors that sometimes need to be used including ECG monitors, oximeters for measuring blood oxygen, glucose meters, and others.

Additionally, many patients are at risk for falls. There is no current way to detect a fall when a patient is by themselves in their room or in the bathroom. In many circumstances, bed alarms and chair alarms are not able to notify a caregiver if a patient falls and needs help.

Another problem that the COVID 19 pandemic brought to light is that demand can increase rapidly in an emergency. And the supply chain can quickly become unstable and experience lengthy delays, which can have draconian effects for healthcare, which means there is a need for less complicated, multifunctional monitors.

### SUMMARY

In one aspect, a medical monitoring device comprises a monitoring unit. The monitoring unit comprises a processor, a transceiver in data communication with the processor, and a power supply in electrical communication with the processor and the receiver. The monitoring device further comprises a plurality of wireless electrodes. Each of the wireless electrode comprises an electrode, a transmitter in electrical communication with the electrode, a power supply in electrical communication with the transmitter, and an adhesive operably connected to the electrode.

In another aspect, an electrocardiogram (ECG) monitoring device comprises a monitoring unit. The monitoring unit comprises a processor, a transceiver in data communication with the processor, and a power supply in electrical communication with the processor and the receiver. The ECG monitoring device further comprises at least 10 wireless electrodes, each of the wireless electrode comprising an electrode, a transmitter in electrical communication with the electrode, a power supply in electrical communication with the transmitter, and an adhesive operably connected to the electrode.

In another aspect, a method of generating an electrocardiogram (ECG) comprises: detecting at least 10 ECG signals through a plurality of wireless electrodes; wirelessly transmitting each of the at least 10 ECG signals from the electrodes to a monitoring unit; and displaying each of the at least 10 wireless signals on a display in real time.

### BRIEF DESCRIPTION OF THE DRAWINGS

FIG. 1 is a top plan view of a patient monitoring device.

FIG. 2 is a top plan view of the wrist mounted patient monitoring device shown in FIG. 1 connected to an oximeter.

FIG. 3 is a diagram illustrating the patient monitoring device illustrated in FIG. 2 in data communication with a portable computing device.

FIG. 4 is a top plan view of the patient monitoring device shown in FIG. 1 with a blood pressure cuff in a mounting strap.

FIG. 5 is a diagram showing positions on a patient's body that the patient monitoring device shown in FIGS. 1 can be worn.

FIG. 6 is a diagram illustrating a hospital network using the patient monitoring device illustrated in FIG. 1.

FIG. 7 is an alternative embodiment of the hospital network illustrated in FIG. 6.

FIG. 8 is another alternative embodiment of the hospital network illustrated in FIG. 6.

FIG. 9 is a block diagram showing an architecture for the patient monitoring device shown in FIG. 1.

FIG. 10 is a block diagram showing an alternative embodiment of the architecture for the patient monitoring device illustrated in FIG. 1.

FIG. 11 is a block diagram showing an alternative embodiment of the architecture for the patient monitoring device illustrated in FIG. 1.

FIGS. 12 and 13 are a top plan view and a side view, respectively, of a wireless ECG electrode.

FIGS. 14 and 15 are a top plan view and a side view, respectively, of a wireless potentiostat.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

For purposes of this patent document, the terms “or” and “and” shall mean “and/or” unless stated otherwise or clearly intended otherwise by the context of their use. Whenever appropriate, terms used in the singular also will include the plural and vice versa. The use of “a” herein means “one or more” unless stated otherwise or where the use of “one or more” is clearly inappropriate. The use of “or” means “and/or” unless stated otherwise. The use of “comprise,” “comprises,” “comprising,” “include,” “includes,” “including,” “having,” and “has” are interchangeable and not intended to be limiting. The term “such as” also is not intended to be limiting. For example, the term “including” shall mean “including, but not limited to.”

All ranges provided herein include the upper and lower values of the range unless explicitly noted. Although values are disclosed herein when disclosing certain exemplary embodiments, other embodiments within the scope of the pending claims can have values other than the specific values disclosed herein or values that are outside the ranges disclosed herein.

Terms such as “substantially” or “about” when used with values or structural elements provide a tolerance that is ordinarily found during testing and production due to variations and inexact tolerances in factors such as material and equipment. These terms also provide a tolerance for variations found in nature and environmental conditions due to factors such as changes in temperature or humidity.

In general terms, this patent document is directed to a medical monitoring device for use in hospitals, healthcare clinics, and remote patient locations. In example embodiments, the medical monitoring device is wearable and includes one or more patient monitors. In example embodiments, the medical monitoring device is wireless and does not have electrical leads or other wires. This configuration increases mobility for the patient and increases reliability because there is less opportunity for electrical leads or wires to become disconnected or otherwise fail. It enables a patient to move around a room more readily or even throughout a healthcare facility. For example, a patient can be moved to another area of the healthcare facility for testing or imaging without having to disconnect the health monitors. It also enables a patient to be comprehensively monitored from home, a rehabilitation facility, or hospice. Another advantage is that eliminating leads and other wires can increase patient comfort. In other embodiments, the patient monitoring device uses a limited number of lead or wires that do not limit movement of the patient in a meaningful way.

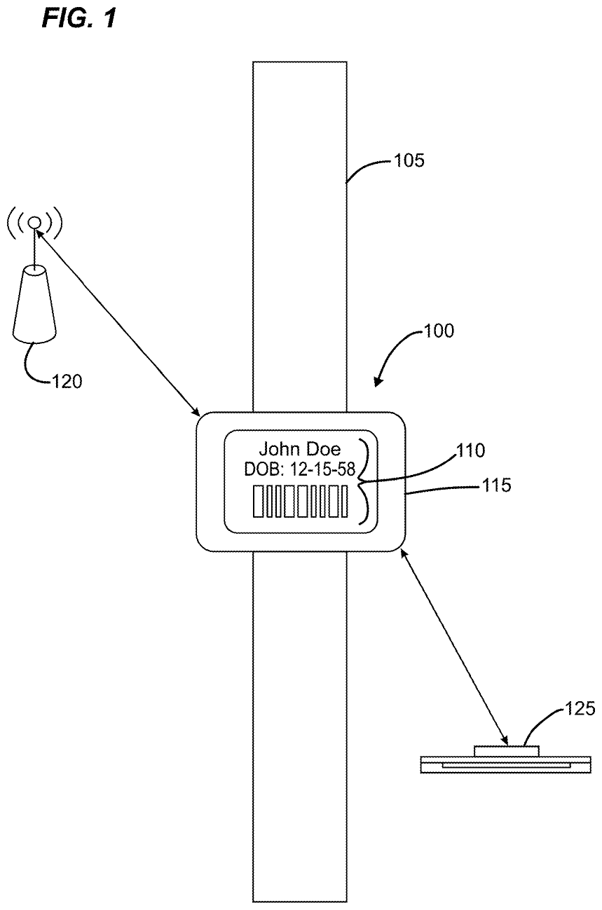

Referring now to FIG. 1, a patient monitoring device 100 includes a housing 115 mounted on a strap 105 that attaches to a patient's body. In example embodiments, the strap is a wrist strap, chest strap, head strap, ankle strap, or any other suitable strap. In yet other embodiments, the patent health monitor has a clip to be attached to the patient's hospital gown or other clothing. In yet other embodiments, the patient monitoring device has an adhesive on its surface so it can adhere to a patient's skin.

The patient monitoring device can have visible ID markings 110 that identify the patient. Examples of visible ID marking include a printed name, printed date of birth, and a scannable bar code that corresponds to the patient ID number such as a clinic or patient number. The bar code can be scanned to access information about the patient such as the patient's medical records. The patient ID marking can be printed on the patient monitoring device or its strap, printed on a sticker attached to the patient monitoring device, or visible on an electronic display such as an LCD display.

The patient monitoring device includes one or more transceivers. At least one of the transceivers is configured to connect to and exchange data with an access point 120 that is connected to a healthcare facility's computer network. In other example embodiments, the patient monitoring device is in radio communication with cellular communication networks, which provides the patient with even greater mobility.

At least one of the transceivers is also configured to communicate with one more sensors such as wireless electrodes 125 and analyte sensors (not shown). The transceivers can be configured to communicate using a suitable communication protocol. Examples include 802.11 protocols (e.g., 802.11a, b, c, g, n, ac, ah, ax, and ad); Bluetooth; IEEE 802.15.1 (WPAN); IEEE 802.15.4 (LR-WPAN); 802.15.5 (mesh networking); 802.15.16 (body area networks); wireless sensor actor networks (WSAN); 6LoWPAN (IPv6—low Power Wireless Personal Area Networks); ANT (wireless sensor network); Zigbee; Ultra-wideband). In at least some examples embodiments provide low power communication that conserves battery energy and that has a sufficient bandwidth to provide fast data communication. In some example embodiments, the bandwidth is sufficient to provide real time data communication.

In example embodiments, the patient monitoring device and communication network operate in real time. These embodiments enable care givers to monitor in real time a patient's clinical measurements of body functions (e.g., pulse rate, blood oxygen levels, blood pressure, respiration rate, ECG data, etc.) and measurement of analytes and other substances (e.g., blood glucose). Real time sensing, measuring, and communication will have some latency, but in general matches human perception of time or are observed as they happen without perceptible delay.

The patient monitoring device can include a variety of sensors and monitors. Examples include electrodes for generating an ECG or monitoring a pulse rate, a pulse oximeter, fall detectors, and glucose sensors. There are many advantages for the patient monitoring device disclosed herein. For example, it eliminates or minimizes the need to tether a patient to large or stationary patient monitors so the patient can move to different rooms or different rooms and areas of the healthcare facility such as imaging or testing while still being monitored. It also eliminates the need for electrical leads for electrodes and analyte sensors. As a result, the patient can move more freely. It also increases the reliability of monitors because there are no electrical leads or wires that can accidentally disconnect.

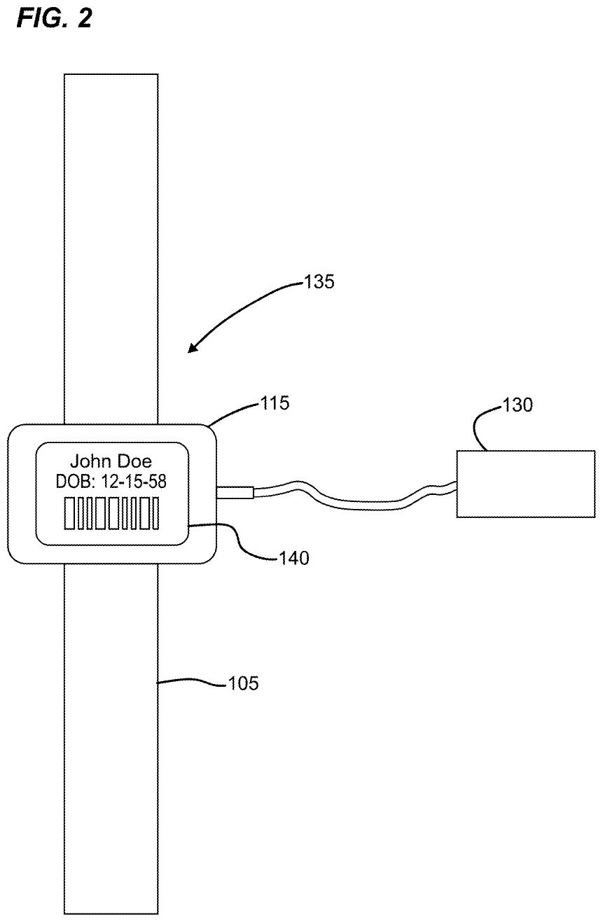

FIG. 2 illustrates an alternative embodiment 135 of the patient monitoring device 100 shown in FIG. 1 and includes a housing 115 and a strap 105. The housing has an electronic display 140 showing a patient name, date of birth and a bar code. Additionally, a pulse oximeter sensor 130 in the form of a clip for attaching to a finger time is connected to the housing via a relatively short electrical lead. For example, the electrical lead might have enough length that it can reach from the clip to the housing mounted on a patient's wrist. Such a configuration does take resources from the transceivers and is short enough that it does not impede movement of the patient or risk being accidentally disconnected from the patient monitoring device. The patient monitoring device is in radio communication with a network access point.

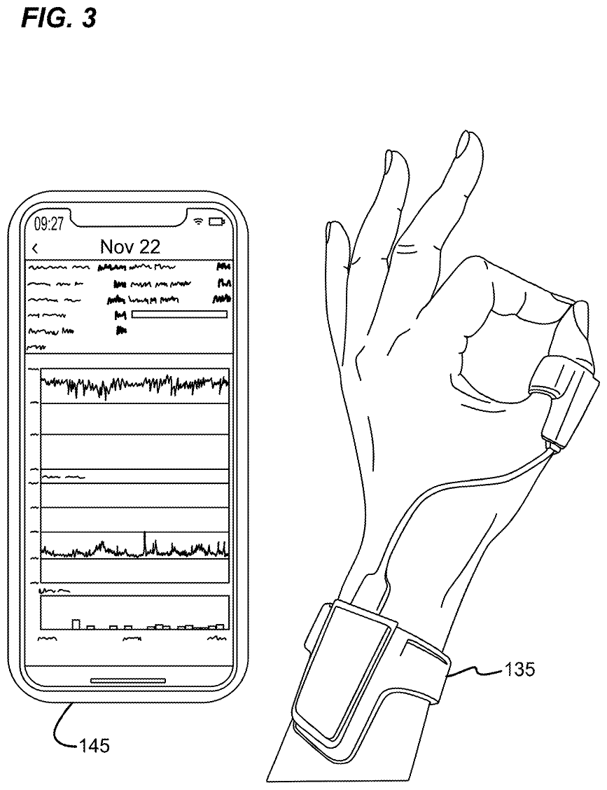

FIG. 3 illustrates the patient monitoring device 135 shown in FIG. 2 and a portable computing device 145 that can be used to view data from the patient monitoring device. The portable computing device can be any type of computing device that a caregiver can handle and can carry with them. Examples include smartphones, tablet computers, and laptop computers. The portable computing device can display output from the monitors. In the illustrated example, the portable computing device is displaying data from the pulse oximeter, a chart of blood oxygen levels over time, pulse rate over time, and the patient's motion over time.

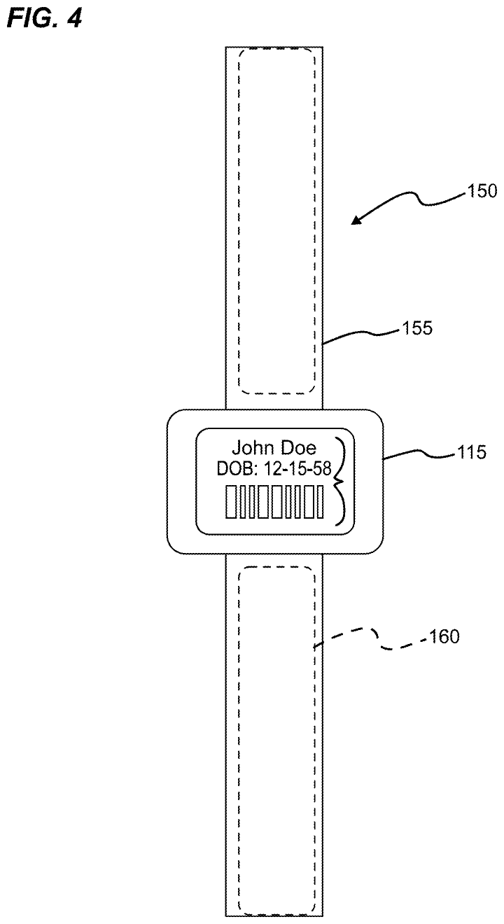

FIG. 4 illustrates an alternative embodiment 150 of the patient monitoring device 100 shown in FIG. 1 and includes a housing 115 and a display 140. The patient monitoring device 150 includes a strap 155 and a bladder 160 positioned within the strap that can be selectively inflated and deflated. The bladder functions as a blood pressure cuff and can be used to measure a patient's blood pressure. In these embodiments, the bladder is sized to accurately measure blood pressure and the patient monitoring device can be placed at a position of the body suitable for measuring blood pressure.

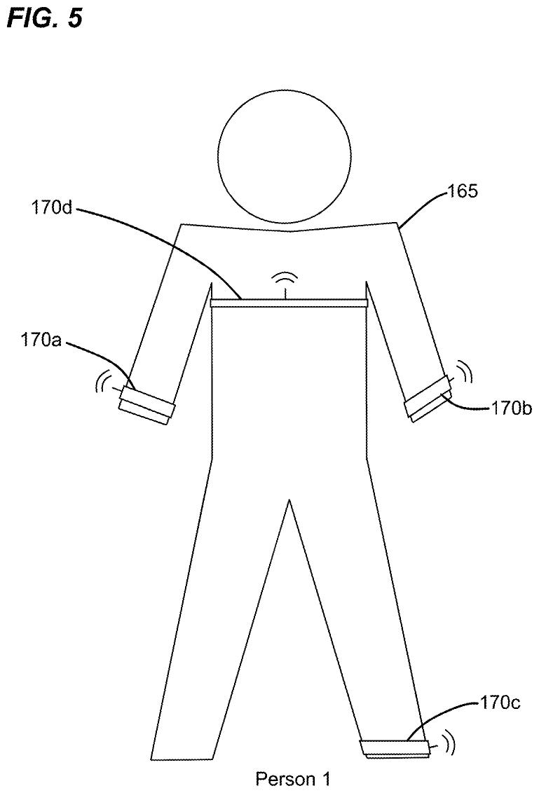

FIG. 5 illustrates a patient 165 and the different locations the patient monitoring device 100 can be worn or positioned in the patient's body. Examples include the left wrist 170a, right wrist 170b, an ankle 170c, and chest 170d. Other positions include the head, upper arms, calf, thigh, and abdomen. The most appropriate position can be dictated by a variety of factors such as the vital signs being measured, patient comfort, and the commercial design of the patient monitoring device.

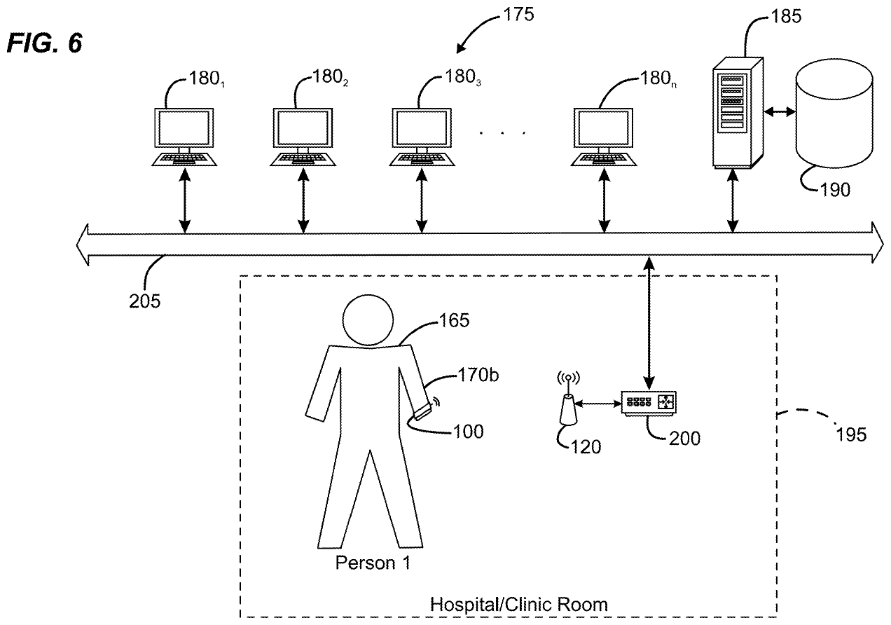

FIG. 6 illustrates a computer network 175 in a healthcare facility. A patient is receiving care in a room 195. The room includes an access point 120 connected to the healthcare facilities' data network 205. The room can be a private or shared hospital room, intensive care unit, room having imaging equipment, lab, lobby, hall, or any other area in a healthcare clinic where a patient might be located. The data network can be any suitable wired or wireless data network. Examples include a local area network (LAN), wireless local area network (WLAN), campus area network (CAN), enterprise private network (EPN), the Internet or cloud, virtual private area network (VPN), or any other suitable local or wide area network.

A one or more computing devices 180₁-180ₙ and servers 185 are in data communication with the data network. The computing devices can be any suitable computing device such as desktop computers, terminals, tablet computers, smart phones, alarm units, or any other device that can receive data over the data network. In at least some example embodiments, the computing devices are network clients that can be located at nursing stations, labs, or in central monitoring facilities. The computing devices can include web browsers capable of receiving and displaying HTML documents including graphics and audio embedded in the HTML document. In these embodiments, the computer terminal can display health information being monitored by the patient monitoring device including pulse rates, ECGs, pulse oxygen, glucose measurements, and other vital signs and biologic measurements. It also can display or sound alarms in the event a measurement made by the patient monitoring device exceeds a threshold level.

The server can be a hospital information management server (HIMS) or some other type of server and is in data communication with a data store 190. The server receives data from the patient monitoring device and stores it in the storage. In at least some example embodiments, the patient monitoring device receives signals from the various sensors (discussed in more detail herein) and relays them to the server. In turn, the server processes those signals to generate a user interface and set alarms. For example, the server can include a web server that generates HTML documents that include plugins for displaying video and playing audio. The HTML documents are transmitted to the computer terminals for display on the monitors.

The terminals 180₁-180ₙ, can be any suitable computing device programmed to receive and process signals from the patient monitor. Examples include desktop PCs, laptop PCs, tablets, and smart phones.

In at least some example embodiments, different terminals can have different access rights. Examples include rights to view or edit patient records and charts; rights to display only certain information such as ECG charts, Pulse Ox, pulse rate, or alarms; and rights to display or edit administrative information such as billing, scheduling, insurance. Additionally, rights to view or edit information can depend on the individual accessing the terminal. For example, physicians, nurses, clinicians, pharmacists, administrators, or other caregivers can have different rights. The rights to view and access certain information also can depend on the location of the terminal. For example, terminals in hospital rooms, nurses, stations, labs and imaging facilities, or third-party service providers can each have different rights to access and edit information.

In example embodiments, the access points can be wired or wireless. They can be indoor or outdoor. They can operate according to any suitable communication protocol. Examples include WiFi 4 (IEEE 802.11n), WiFi5 (IEEE 802.11ac), WiFi 6 (IEEE 802.11ax), WiFi 6E ((IEEE 802.11ax).

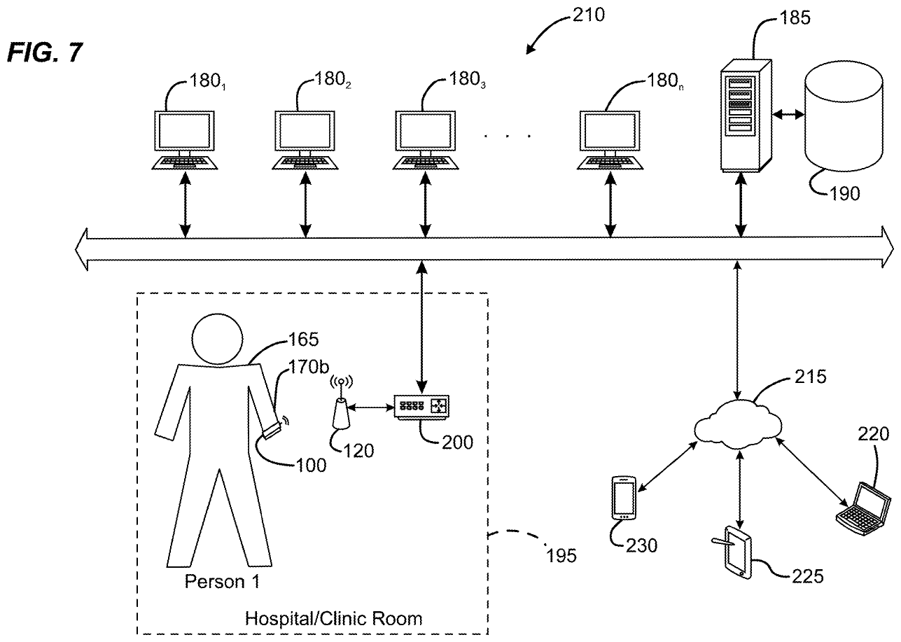

FIG. 7 illustrates an alternative embodiment 210 of the patient monitoring network, which is substantially similar to the embodiment 175 shown in FIG. 6 and includes a local area network, terminal, access points. Additionally, the patient monitoring network is connected to the cloud or other wide area network 215. A user can connect to the server or patient monitoring device remotely through the cloud using a computer 220, tablet 225, or smart phone 230. This embodiment enables users or third-party service providers to monitor a patient's vital signs or other health parameters remotely. It also enables patient wearing a patient monitoring device to be monitored from a remote location.

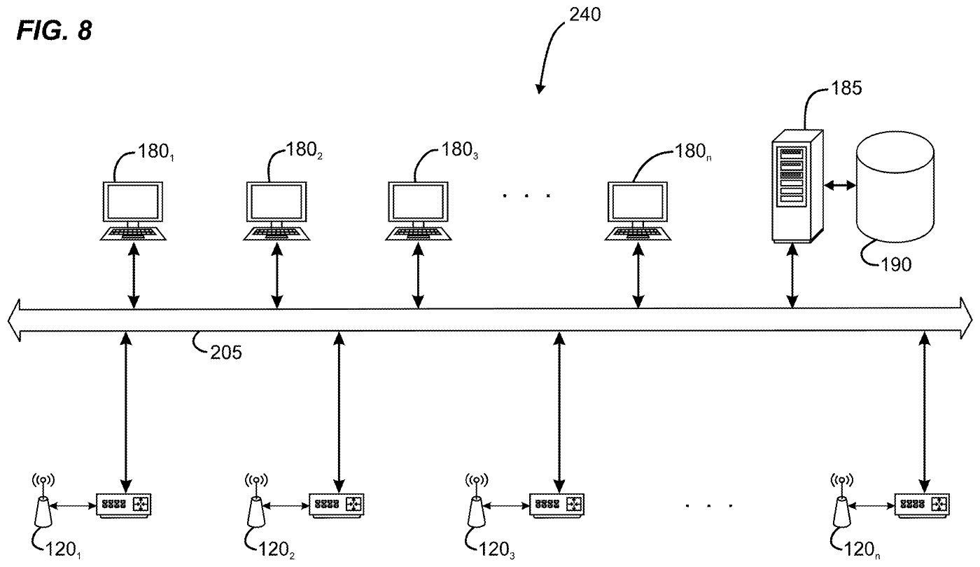

FIG. 8 illustrates another alternative embodiment 240 of the patient monitoring network 175, which includes the local area network, terminals, server, and storage. It also includes a plurality of access points 120₁-120ₙ that can be positioned at various locations in the healthcare facility. For example, access points can be positioned in rooms such as hospital rooms, examination rooms, imaging rooms, surgical rooms, labs, lobbies, hallways, outdoor courtyards, cafeterias, other common areas, or any other location where a patient might go. As a patient moves around the care facility, the patient monitor hands off its data connection from one access point to another. An advantage of this embodiment is that the patient's vital signs and other physical parameters can be monitored as the move around the facility. Another advantage is that the location and movement of the patient always can be determined.

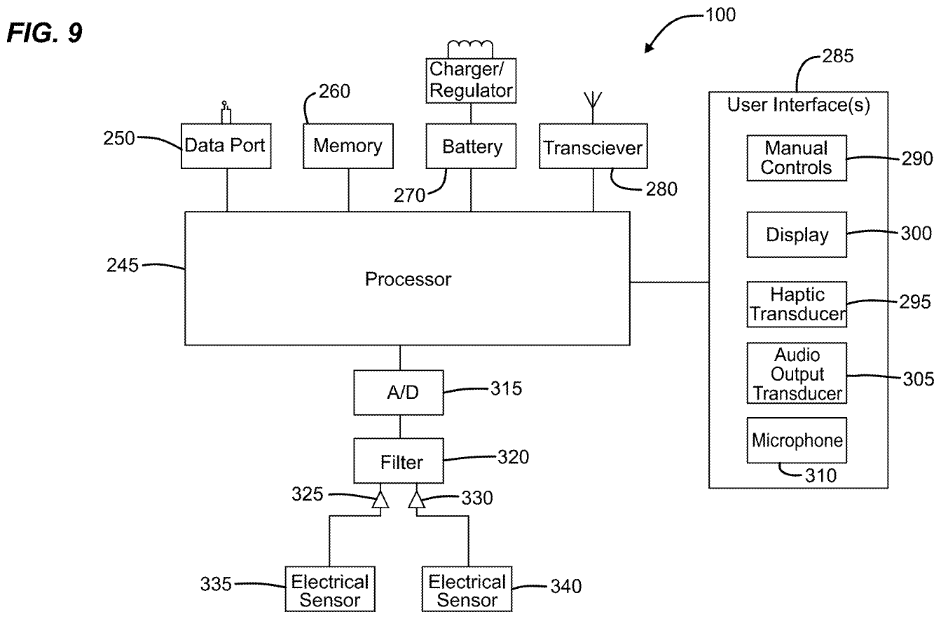

FIG. 9 illustrates the components of the patient monitor 100. It includes a processor 245, memory 260, data port 250, a transceiver 280, a battery 270, one or more electrical sensors 335 and 340, and one or more user interfaces 285. The processor can be any circuit configured for processing signals related to the monitoring a patient and can include one or more processing units. A processing unit is a physical device or article of manufacture comprising one or more integrated circuits that selectively execute software instructions. In various embodiments, the processing system is implemented in various ways. For example, the processing system can be implemented as one or more processing cores. In another example, the processing system can include one or more separate microprocessors or microcontrollers. In yet another example embodiment, the processing system can include an application-specific integrated circuit (ASIC) that provides specific functionality. In yet another example, the processing system provides specific functionality by using an ASIC and by executing computer-executable instructions.

The memory device may include one or more internally fixed storage units, removable storage units, and/or remotely accessible storage units. The various storage units may include any combination of volatile memory and non-volatile memory. Examples of storage units include random access memory (RAM), read only memory (ROM), and EEPROM. The storage units may be configured to store any combination of information, alarm limits, data, instructions, software code, etc.

Data ports can include any physical data port suitable for serial data communication, although alternative embodiments can use a data port for parallel data communication. Examples of wired data ports include USB Type A, USB Type B, USB Type C, Mini USB, Micro USB, Lightning, RS-232, Ethernet, or any other type of data port for serial data communication. The type of wired data port connector can depend on compatibility with the computers and other hardware with which the patient monitor will be connected. It also will depend on the communication protocol that the patient monitor and its associated networking and data processing equipment use. Examples of communication protocols include USB 1.x, USB 2.0, USB 3.X, USB4, RS-232, and Ethernet. In at least some embodiments, the data port connector can carry power used to power the patient monitor and charge the batter.

The transceiver is electrically connected to an antenna. In at least some embodiments, the antenna is internal and positioned within the patient monitor housing. The transceiver can use any suitable wireless communication protocol such as TCP/IP. Examples of TCP/IP protocols include 802.11a, 802.11b, 802.11, g, 802.11n (WiFI 4), 802.11ac (WiFi 5), 802.11ax (WiFi 6), and Bluetooth. In example embodiments, the patient monitor wirelessly connects to the access points and hence the local area network through the transceiver. In example embodiments, the patient monitor also can receive or send data with electrical sensors and other peripheral devices through the transceiver. Examples of such peripheral devices include medical instruments such as analyte sensors, temperature sensors, and blood pressure cuffs. Other examples include computers and smart phones.

The electrical sensors include electrodes that sense electrical potential at the skin surface that is caused by the electrical current that controls the heart and then propagates to the skin. The signal generated at the sensors passes through amplifiers 325 and 330 and the filter 320 to remove noise and then through the analog-to-digital (A/D) converter 315. After the signal is digitized at the A/D converter it is input to the processor for processing.

In example embodiments, the battery is a rechargeable battery. It is in electrical communication with a charger and power regulator. In at least some embodiments, the charger also is in electrical communication with an inductive coil for wirelessly charging the battery. The wireless charging can operate according to any suitable standard such as the QI wireless charging standard. In alternative embodiments, the charger/power regulator is in electrical communication with the data port and can receive power for charging the battery through a wired cable such as a USB cable.

In example embodiments, the user interface can include one or more user interfaces such as an electronic display 300, haptic transducer 295, audio output transducer 305, or microphone 310. Examples of electric displays include LCD displays, color or black and white displays, touch screens, and e-ink displays. An advantage of an e-ink display is that is requires reduced or no power to maintain an image, which conserves batter power. The display, especially an e-ink display is useful for displaying static information such as the patient's name, date of birth, clinic number, and bar codes or QR codes. The display also can be used to display monitored information such patient pulse, blood oxygen level, and body temperature.

The haptic transducer can be used to deliver information through tactile senses. It might be useful to notify the patient or reminders, alters, or alarms. Examples of audio output transducers include speakers and buzzers. A speaker could be useful to deliver messages, whether pre-recorded or a live message. The speaker and microphone could be used for remote voice communication with a caregiver or other person. The user interface also can include manual controls such as buttons, sliders, or dials.

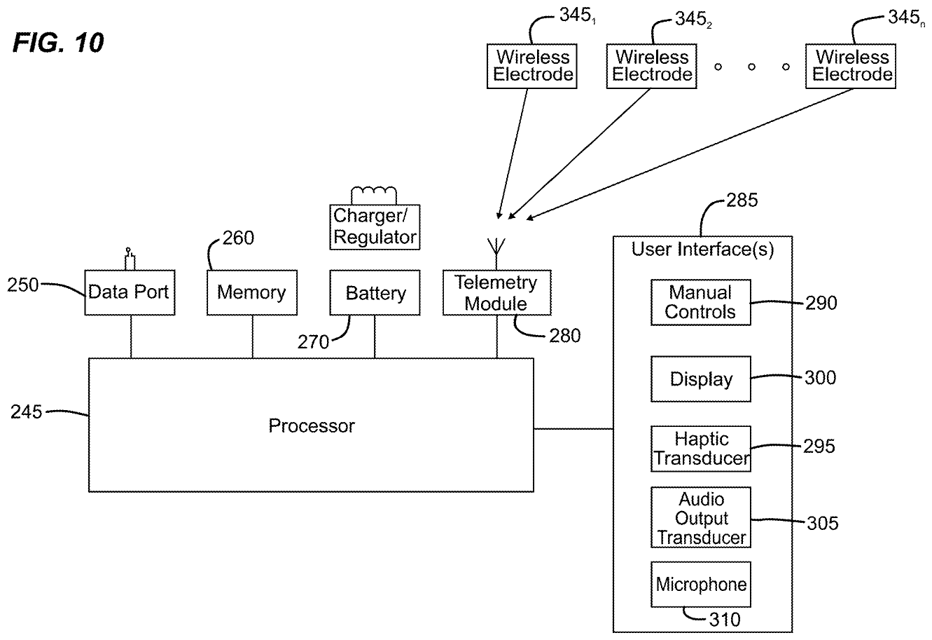

FIG. 10 illustrates the architecture for a patient monitoring system that is substantially similar to the patient monitor illustrated in FIG. 9 and includes a patient monitor having a processor, memory, a data ports, a transceiver, and a battery. In this embodiment, ECG sensors 345₁-345ₙ are not physically attached to the patient monitor but are in data communication with the transceiver.

An advantage of this embodiment is that the patient monitoring system can have as many ECG electrodes are necessary. An example embodiment has 2 electrodes. Other examples embodiments have 3, 5, or 10 ECG electrodes. Other embodiments have between 2 and 12 ECG electrodes. An advantage of having more ECG electrodes is that the monitor can generate a more accurate ECG. Alternatively, reducing the number of electrodes can reduce expense and patient discomfort.

Another advantage of wireless ECG electrodes is they eliminate any physical constraints imposed by the housing and physical structure of the health monitor. The elimination of electrodes is especially significant for ECG using 10 electrodes, which commonly use 12 leads. It is easier for a caregiver to set up. Additionally patient mobility and comfort is not hindered by the leads.

Another alternative embodiment still includes one or more wireless electrodes and one or more electrodes mounted in the housing of the patient monitor and hardwired in the patient monitor's circuit in FIG. 9. The hardwired electrode also can be used to generate an ECG, can be used to measure the patient's pulse, or can be used for any other suitable reason.

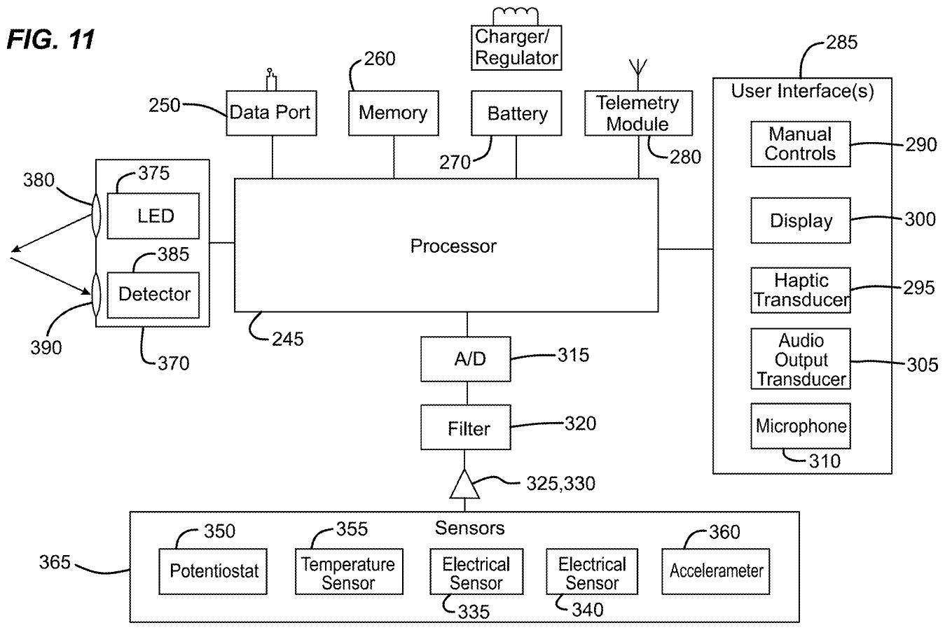

The patient monitor illustrated in FIG. 11 is substantially similar to the patient monitor illustrated in FIG. 9 and includes a processor, data port, memory, battery, charger/regulator, transceiver and antenna, user interfaces, including manual controls, a display, haptic transducer, audio output transducer, and microphone; A/D converter, filter, amplifier, and sensors including ECG electrodes. The patient monitor also includes one or more additional sensors 365 such as a potentiostat 350, temperature sensor 355, and accelerometer 360. Signals output by the additional sensors are also amplified by the amplifier, filtered, and then converted to digital signal before they are input to the processor for processing.

In example embodiments, the potentiostat detects or measures specific analytes in biological fluids collected from the patient. Examples of analytes and other metabolites that can be detected and measured include blood glucose, cholesterol or lactate, ions (e.g., K+, Na+, Ca 2+), and metals (e.g., zinc, lead).

The temperature sensor can be any sensor suitable for medical applications. Examples include thermistors, infrared sensors, thermocouples, thermometers, resistance temperature detectors (RTD's), and semiconductor temperature sensors.

The accelerometer can include any accelerometer suitable for sensing movement. Example embodiments can include three accelerometers. The accelerometers can be positioned at angle to one another. For example, the accelerometers can be orthogonal to one another such as along a x-, y-, and z-axes. In other embodiments, one or more of the accelerometers can have a skewed orientation relative to one or more of the other accelerometers. An advantage of skewed orientations is that there is greater flexibility in packaging the accelerometers in the housing. Other embodiments might have more or less than three accelerometers such as one accelerometer or two accelerometers. An advantage of the accelerometers is that they can be used to detect when a patient falls. For example, the processor can programmed to monitor outputs from the accelerometers and determine if there was a fall when the output matches a defined pattern such as a rapid downward movement, movement above a threshold rate, a sudden stop in movement, other possible movement characteristics, and combinations of certain movement characteristics. In example embodiments, the accelerometers can be used for other purposes such as detecting movement of a patient to aid in locating the patient or detecting certain movements of the patient to diagnose movement disorders.

The health monitoring device also includes a pulse oximeter 370 in data communication with the processing unit. The pulse oximeter includes a light emitting diode (LED)375 or other light source and a corresponding transmission lens 380 for focusing light into tissue of the patient. The pulse oximeter also comprises a photo detector 385 and a corresponding receiving lens 390. The receiving lens receives light reflects from the patient tissue and focuses it onto the photo detector. In example embodiments, the pulse oximeter can be housed in a finger clip that is positioned on the fingertip of the patient when in use. The pulse oximeter can be wired and connected to the patient monitor device through the data port. In other example, embodiments, the pulse oximeter is in data communication with the patient monitor through the transceiver. In these alternative embodiments, the pulse oximeter includes a battery, a transmitter or transceiver, a processor and memory.

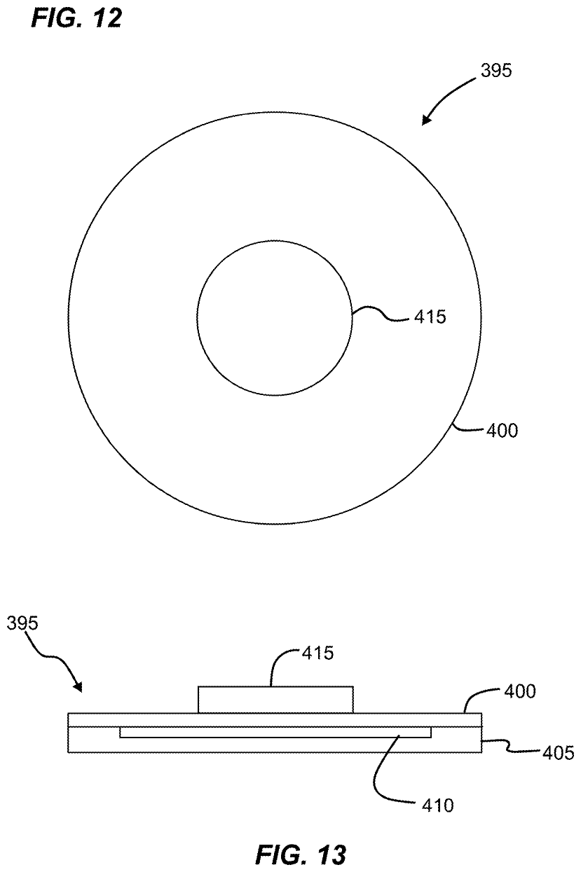

FIGS. 12 and 13 illustrate a wireless ECG electrode 395, which includes a substrate 400, an adhesive layer 405 mounted on the substrate, and an ECG electrode 410 positioned between the substrate and the adhesive layer. An electronic module 415 is positioned on the substrate on an opposite side from the adhesive layer. The adhesive layer is formed with any type of adhesive that is biocompatible, electrically conductive, and will securely hold the wireless electrode against the patient's skin, but still peel from the skin without significant discomfort. In example embodiments, the adhesive also is waterproof or water resistant so it will stay secure in the shower or bath.

The electrode is formed of an electrically conductive material. In example embodiments, the electrode and substrate are flexible so they can more easily conform to the shape of a person's skin at the location of contact. In example, embodiments, the ECG electrode can include one electrode. In other example embodiments, the ECG electrode can have two or more electrodes.

The electronic module includes a battery, processor, memory, transmitter, amplifier for amplifying electrical signals detected from the patient's body, a filter for removing noise from the amplified signal, and an A/D converter for digitizing the amplified and filtered signal. The processor processes the signal and then transmits the data to the patient monitor. In example, embodiment, the electrode has an address, which may be stored in the memory. The processor in the ECG electrode includes the sensed data and address in a word or packages them in a data envelope for transmitting to the patient monitoring device so it can identify the electrode that generated the ECG data. In an alternative embodiment, the patient monitoring devices has a log of the ECG electrodes and their addresses and then polls each one to retrieve the ECG data.

In example embodiments, the entire ECG electrode is disposable. In alternative example embodiments, the electronic module is detachably connected to the substrate and can be used with other substrates, electrodes. adhesive assemblies. In yet other example embodiments, the adhesive layer is a double-sided adhesive that can be removed from the substrate. In this embodiment, a caregiver can remove the adhesive layer, sterilize the ECG electrode, and then apply a new adhesive layer for use with a different patient.

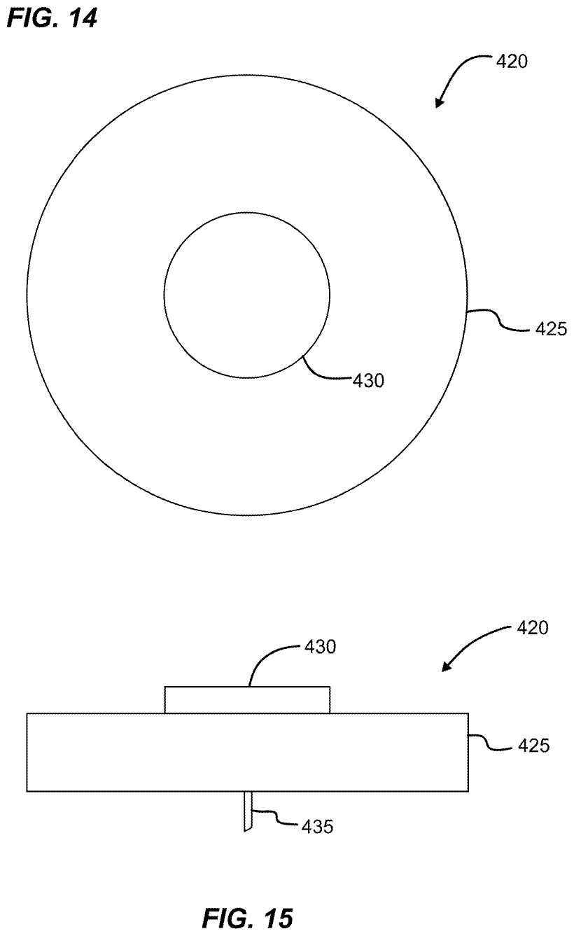

FIGS. 14 and 15 illustrate an example embodiment of a potentiostat 420. The potentiostat had a housing 425, an electronic module 430, and a needle 435 for collecting biological fluids from the patient. The housing defines a cavity that is in fluid communication with a lumen defined in the needle. The cavity hold fluid extracted through the needle for testing. Additionally, electrodes are at least partially positioned in the cavity for testing fluids. Alternatively, or in addition to the electrodes, a light source and photo detector are positioned to transmit light into the fluid sample held in the cavity.

The electronic module includes a battery, processor, memory, transmitter, amplifier for amplifying electrical signals detected from the patient's body, a filter for removing noise from the amplified signal, and an A/D converter for digitizing the amplified and filtered signal. The processor processes the signal and then transmits the data to the patient monitor. In example embodiments, the electrode has an address, which may be stored in the memory.

In at least some example embodiments, the output signals from the ECG electrodes can be processed by a learning system to generate an electrocardiogram. The learning system is an electronic processing system that includes one or more processing units that can be located in the processing unit of the patient monitoring device, a server on the healthcare network, or on a server of a third-party health care or analytics service provider, od distributed between two or more locations or entities. In example embodiments, the learning system is programmed with and executes machine-learning algorithms to analyze the ECG or movement data. In example embodiments, the learning system comprises one or more neural networks. The neural network can be an artificial neural network. Other example embodiments can include other configurations of circuits, processors, and algorithms for analyzing the movement data.

In example embodiments, the neural network includes a plurality of input nodes, a plurality of output nodes, and a plurality of processing nodes between the input and output nodes. For generating an ECG or setting alarms, each input node corresponds to information related to the electrical signal from the ECG electrode, examples include the position of the electrode and parameters defining the electrical signal. Examples of parameters that might be input to the neural network include sampling rate, amplitude, current, period, frequency, phase, and voltage level. When the neural network is trained, the output is then processed to determine a final data point for generating the ECG for display and recording. In at least some example embodiments, the trained neural network compares data from the output node to a database to determine the final data point.

An advantage of using a neural network to generate an ECG is that as the neural network is trained it will generate more and more accurate ECSs. This can have any advantage for generating accurate ECGs when using fewer electrodes, possible as few as two electrodes.

At least some example embodiments also use neural networks from processing the accelerometers to identify events such as fall or to identify movement disorders. In these embodiments, the output parameters fed into the input nodes can include the identity of the accelerometer, the angular orientation of the accelerometer relative to other accelerometers in the system, the sampling rate, and the magnitude of the signal output by the accelerometer. Again, data from the output nodes are input to a data base to identify if there was a fall or identify other information that might be helpful to a caregiver.

The various embodiments described above are provided by way of illustration only and should not be construed to limit the scope of the ideas embodied in this patent specification. Those skilled in the art will readily recognize various modifications and changes that may be made without following the example embodiments and applications illustrated and described herein, and without departing from the true spirit and scope of the ideas embodied in this disclosure. It is intended that any such modifications and equivalents be included in the scope of this patent application.

1. A medical monitoring device comprising:
a monitoring unit, the monitoring unit comprising a processor, a transceiver in data communication with the processor, and a power supply in electrical communication with the processor and the receiver; and
a plurality of wireless electrodes, each of the wireless electrode comprising an electrode, a transmitter in electrical communication with the electrode, a power supply in electrical communication with the transmitter, and an adhesive operably connected to the electrode.

2. The medical monitoring device of claim 1 wherein the plurality of wireless electrodes comprises 10 wireless electrodes.

3. The medical monitoring device of claim 1 wherein the plurality of wireless electrodes comprises 12 wireless electrodes.

4. The medical monitoring device of claim 1 further comprising a display in data communication with the monitoring unit.

5. The medical monitoring device of claim 4 wherein:
each of the wireless electrodes detect an electrocardiogram signal (ECG) from electrical activity of a patient's heart when attached to a patient; and
the monitoring unit is programmed and configured to receive the ECG signal from each of the wireless electrodes and transmit each of the ECG signals to the display.

6. The medical monitoring device of claim 5 wherein the ECG signals are displayed on the monitor in real time relative to when they are detected by the wireless electrodes.

7. The medical monitoring device of claim 5 wherein:
the monitoring unit is in wireless data communication with an access point; and
the access point is in data communication with a hospital local area network (LAN).

8. The medical monitoring device of claim 7 wherein the LAN is in data communication with a hospital information management server (HIMS) and the monitoring device is programmed to communication the ECG signal to the HIMS.

9. The medical monitoring device of claim 7 wherein the monitoring unit is located in one room of a building and the display is located is a different room of the building.

10. The medical monitoring device of claim 1 further comprising a wireless pulse oximeter, the pulse oximeter being in wireless communication with the monitoring unit.

11. An electrocardiogram (ECG) monitoring device comprising:
a monitoring unit, the monitoring unit comprising a processor, a transceiver in data communication with the processor, and a power supply in electrical communication with the processor and the receiver; and
at least 10 wireless electrodes, each of the wireless electrode comprising an electrode, a transmitter in electrical communication with the electrode, a power supply in electrical communication with the transmitter, and an adhesive operably connected to the electrode.

12. A method of generating an electrocardiogram (ECG), the method comprising:
detecting at least 10 ECG signals through a plurality of wireless electrodes;
wirelessly transmitting each of the at least 10 ECG signals from the electrodes to a monitoring unit; and
displaying each of the at least 10 wireless signals on a display in real time.

13. The method of claim 12 wherein displaying each of the at least 10 wireless signal comprises wireless transmitting the ECG signals from the monitoring unit.

14. The method of claim 13 wherein the monitoring unit is in data communication with a local area network, and displaying each of the at least 10 wireless signal comprises transmitting the ECG signals from the monitoring unit, through an access point, and to the display.
